(19) United States
(12) Patent Application Publication
(10) Pub. No.: US 2026/0306396 A1
(43) Pub. Date: Oct. 8, 2026

(54) COSMETIC USE AS AN EXFOLIATING AGENT OF AN ETHANOLIC EXTRACT OF SHELLS OF CASHEW NUTS

(71) Applicant: EPHYLA, ARZAL (FR)

(72) Inventor: Vincent BOURGETEAU, FEREL (FR)

(21) Appl. No.: 19/640,021

(22) Filed: Apr. 6, 2026

(30) Foreign Application Priority Data
Apr. 7, 2025 (FR) FR2503636

(51) Int. Cl. A61K 8/9789; A61K 8/06; A61K 8/25; A61K 8/368; A61K 8/92; A61Q 19/00; A61Q 19/02

(52) U.S. Cl. CPC A61K 8/9789; A61K 8/062; A61K 8/25; A61K 8/368; A61K 8/922; A61Q 19/00; A61Q 19/02; A61K 2800/28; A61K 2800/805

(57) ABSTRACT

Extract of shells of cashew nuts are obtained by a method of preparation including at least the following stages, in the following order: providing shells of cashew nuts, grinding the shells of cashew nut shells to an average particle size of 5 mm or less, extracting from the shells of cashew nuts using ethanol at 96° at a temperature between 20° C. and 45° C., filtering, recovering the filtrate, evaporating the ethanol, recovering the extract obtained or a composition including the extract as an epidermis exfoliating agent.

### CROSS-REFERENCE TO RELATED APPLICATIONS

This application claims priority to French Patent Application No. 2503636, filed Apr. 7, 2025, the entire content of which is incorporated herein by reference in its entirety.

### FIELD

The present invention relates to the field of epidermis exfoliation, also known as epidermabrasion. More precisely, the invention relates to an extract of shells of cashew nuts and the use of such extract in cosmetics. The product is applied to the skin, as an epidermis exfoliating agent.

### BACKGROUND

The skin is the main barrier of protection of the human body against external aggressions such as air pollution, weather change and UV radiation.

The skin consists of three layers: epidermis, dermis and hypodermis. The epidermis, which is the superficial layer in contact with the exterior, is more particularly concerned by interactions with the exterior environment.

The epidermis is covered by a hydrolipidic film and is composed of four layers: stratum corneum (horny layer), stratum granulosum (granular layer), stratum spinosum (spiny layer) and stratum basale (basal layer). The epidermis normally consists of four types of cells: keratinocytes which represent 80% of all epidermis cells, the remaining 20% being composed of melanocytes, Langerhans cells or immunocompetent cells and of Merkel cells, these three groups of cells being dispersed between the keratinocytes.

The skin continuously renews and regenerates via a process called cell renewal. Epidermal cell renewal eliminates dead skin cells and replaces same with newly formed cells. In addition to giving the skin a youthful and fresh look, cell renewal also contributes to maintaining smooth, healthy and uniform skin.

There are different techniques to stimulate cell renewal such as laser treatments, mechanical (micro) dermabrasion treatments or chemical peels also called “chemical dermabrasions”.

Fruit acids (AHAs) are the main active ingredients used in the cosmetic field to implement chemical peels.

### SUMMARY

A problem addressed which at the origin of an aspect of the present invention, was to propose novel cosmetic compositions based on natural products which have epidermis exfoliating properties.

The inventor has shown that a solution to said technical problem can be provided by means of a particular extract of shells of cashew nuts.

This particular extract of cashew nuts, or “Anacardium Occidentale L” can be used for implementing controlled abrasion or exfoliation of the upper layers of the epidermis.

Unlike acid peel compositions, the extract of shells of cashew nuts has the benefit of not needing to be placed in an acidic medium to be active even if same is still effective at acidic pH. Consequently, the use of the extract of cashew nuts according to the present invention makes it possible to limit or even prevent irritation and discomfort from tingling or from abrasion. Sensitive and reactive skin thus finds a scrub solution that acts delicately to preserve the comfort of the treatment.

According to a first aspect, the present invention is aimed at the cosmetic, non-therapeutic, topical use of an extract of shells of cashew nuts or “Anacardium Occidentale L” as an epidermis exfoliating agent, where said extract of shells of cashew nuts or “Anacardium Occidentale L.” can be obtained by a method preparation comprising at least the following steps, in the following order:
providing shells of cashew nuts,
grinding the shells of cashew nuts to an average particle size of 5 mm or less,
extracting from shells of cashew nuts using ethanol at 96° at a temperature between 20° C. and 45° C.,
filtering,
recovering the filtrate,
evaporating the ethanol,
recovering the extract obtained.

In an embodiment, there is provided a cosmetic, non-therapeutic method of exfoliating the epidermis of a subject, comprising topically applying to the skin of the subject a cosmetically effective amount of an extract of shells of cashew nuts or “Anacardium Occidentale L.”, wherein said extract of shells of cashew nuts is obtained by a method of preparation comprising at least the following steps, in the following order:
providing shells of cashew nuts,
grinding the shells of cashew nuts to an average particle size of 5 mm or less,
extracting from the ground shells of cashew nuts using ethanol at a concentration of about 96° v/v at a temperature between 20° C. and 45° C. to obtain a resulting mixture,
filtering the resulting mixture,
recovering the filtrate,
evaporating the ethanol from the filtrate,
recovering the extract obtained.

According to a second aspect, the present invention is aimed at a cosmetic, non-therapeutic use by topical route, of a composition comprising from 0.1 to 49.9% by weight of said extract of shells of cashew nuts as an epidermis exfoliating agent.

In an embodiment, there is provided a cosmetic, non-therapeutic method of exfoliating the epidermis of a subject, comprising topically applying to the skin of the subject a cosmetic composition comprising from 0.1 to 49.9% by weight of extract of shells of cashew nuts or “Anacardium Occidentale L.” with respect to a total mass of the composition, wherein said extract is prepared by the method according to an embodiment of the invention.

According to a third aspect, the present invention is aimed at a cosmetic, non-therapeutic use by topical route, of an extract of shells of cashew nuts or composition comprising the extract of shells of cashew nuts as epidermis exfoliating agent, used to erase hyperpigmentation spots from the skin and/or to improve skin radiance and/or to impart smoother, lighter and/or more uniform appearance to the skin.

According to a fourth aspect, the present invention is aimed at a cosmetic, non-therapeutic method of treatment for exfoliating the epidermis comprising at least one step of applying to the skin said extract of shells of cashew nuts or said composition according to the invention comprising said extract of shells of cashew nuts.

In an embodiment, there is provided a cosmetic composition for exfoliating the epidermis, comprising:
(a) from 0.1% to 49.9% by weight of an extract of shells of cashew nuts (“Anacardium Occidentale L.”), relative to the total weight of the composition, wherein said extract is prepared by a method comprising: grinding shells of cashew nuts to an average particle size of 5 mm or less; extracting from the ground shells using ethanol at a concentration of about 96% v/v at a temperature between 20° C. and 45° C.; filtering; recovering the filtrate; evaporating the ethanol; and recovering the extract; and
(b) at least one cosmetically acceptable vehicle.

By clearing old cells, including pigmented ones, from the skin, the extract and composition used according to the present invention enhance cell renewal. However, cell renewal contributes to attenuating discolorations of the skin such as age spots and freckles, thereby giving the skin a more uniform complexion.

With aging, the natural cell renewal of the skin slows down. By stimulating such process, fine lines and wrinkles attenuate, which rejuvenates the appearance of the skin.

Furthermore, the cosmetic compositions according to the invention are stable, non-irritating, non-toxic and non-allergenic for the skin.

Other aspects, benefits and properties of the present invention are presented in the following description and examples.

### DETAILED DESCRIPTION

### Definitions

In the present text, unless otherwise specifically indicated, the percentages are expressed by weight of a reference composition.

In the present text, intervals are defined in abbreviated form in order not to need to describe each and all values in the interval. However, any appropriate value in the interval can be chosen as the upper value, lower value, or the end values of the interval. For example, a range of 0.1 to 1.0 represents the end values of 0.1 and 1.0, and the intermediate values of 0.2; 0.3; 0.4; 0.5; 0.6; 0.7; 0.8; 0.9 and all the intermediate intervals between 0.1 to 1.0, such as 0.2 to 0.5; 0.2 to 0.8; 0.7 to 1.0, etc. Unless otherwise specified, an interval defined as “between the value A and the value B” includes the values A and B and is thus equivalent to an interval “between the value A and the value B”, the expression “at least” comprises the value stated after, e.g. “at least 5%” should be understood as also comprising “5%”, the expression “a maximum of” comprises the value stated after, e.g. “a maximum of 5%” should be understood as also comprising “5%”.

Furthermore, in the present text, measurable values, such as a quantity, should be understood as comprising the standard deviations that can be readily determined by a person skilled in the art, in the reference art. For example, such values are intended to comprise variations of +5%.

“Skin” refers to the epidermis of the face or of the body or of the scalp.

In the present text, the expressions “exfoliating effect”, “peeling effect”, “epidermabrasion effect” and “scrubbing effect” are used equivalently.

“Subject” as used herein refers to a human being or animal to whose skin the extract or composition is topically applied.

“Cosmetically effective amount” as used herein refers to an amount of the extract or composition sufficient to produce a detectable exfoliating effect on the epidermis without causing necrotic damage to dermal tissue. The cosmetically effective amount will vary with the concentration of the extract in the composition, the formulation type (leave-on vs. rinse-off), and the duration of application, as described herein.

The compositions according to the present invention are cosmetic, non-therapeutic compositions. Cosmetic compositions are intended to be applied to healthy skin, to treat the epidermis. The cosmetic compositions are as per EC Regulation 1223/2009.

Anacardic acid is a mixture of a plurality of molecules with the following structure: a ring corresponding to salicylic acid comprising a radical —CH₂—R, the radical R being a C₁₄ hydrocarbon radical comprising from 0 to 3 [degrees of] unsaturation. In particular, anacardic acid comprises the following four derivatives of 6-alkyl salicylic acid of formula I:

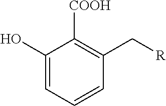

| number of C, | |
|---|---|
| number of | |
| [degrees of] | |
| unsaturation | |
| (on the CH2R | |
| radical) | Name |
| C15, 0 | 6-n-pentadecyl salicylic acid |
| C15, 1 | 6-[8(Z)-n-pentadecenyl] salicylic acid |
| C15, 2 | 6-[8(Z),11(Z)-n-pentadecadienyl] salicylic acid |
| C15, 3 | 6-[8(Z),11(Z),14(Z)-n-pentadecatrienyl] |
| | salicylic acid |

More particularly, anacardic acid can be obtained from shells of cashew nuts.

### Mechanism of Action

Without wishing to be bound by theory, the exfoliating activity of the cashew shell extract is believed to be attributable primarily to its anacardic acid content. Anacardic acid is a class of long-chain alkyl derivatives of salicylic acid. Like salicylic acid, anacardic acid exhibits keratolytic activity.

The C15 alkyl side chains of anacardic acid enhances penetration into the intercellular lipid matrix of the stratum corneum. Importantly, the exfoliating activity is achievable at pH values above 4, including neutral and mildly acidic pH, without the irritation associated with AHA peels. This targeted exfoliation—limited to the epidermis without dermal damage—was confirmed histologically in Example 1.2, which demonstrated detachment of epidermal cells and an intact stratum corneum after 240 minutes of exposure, with no necrotic dermal damage observed.

### Extract of Shells of Cashew Nuts

The extract of shells of cashew nuts or Anacardium occidentale L. used in the present invention can be obtained by a method of preparation comprising at least the following steps, in the following order:
providing shells of cashew nuts,
grinding the shells of cashew nuts to an average particle size of 5 mm or less,
extracting from shells of cashew nuts using ethanol at 96° at a temperature between 20° C. and 45° C.,
filtering,
recovering the filtrate,
evaporating the ethanol,
recovering the extract obtained.

More particularly, the extract of shells of cashew nuts comprises from 60 to 95% by weight of anacardic acid relative to the total weight of the extract. Said extract is thus a concentrated extract of anacardic acid.

Furthermore, the extract of shells of cashew nuts comprises 50 to 90% by weight of 6-[8(Z)-n-pentadecenyl] salicylic acid relative to the total weight of the extract.

### Method of Preparation of the Extract

As already mentioned, the extract can be prepared by a method comprising at least the following steps, in the following order:
providing shells of cashew nuts,
grinding the shells of cashew nuts to an average particle size of 5 mm or less,
extracting from shells of cashew nuts using ethanol at 96° v/v (approximately 192 U.S. proof) at a temperature between 20° C. and 45° C.,
filtering,
recovering the filtrate,
evaporating the ethanol,
recovering the extract obtained.

The extraction may be carried out at a temperature of from 35° C. to 45° C., and for example at approximately 40° C., as illustrated in the working examples herein. Temperatures in the lower portion of the claimed range (20° C. to 35° C.) may be used; however, such lower temperatures may result in reduced extraction yield or may require an extended extraction time to achieve comparable yield. The upper limit of 45° C. is relevant: temperatures exceeding 45° C. promote decarboxylation of anacardic acid to cardanol, which would alter the composition and potentially reduce the exfoliating activity of the extract

### Composition

An aspect of the present invention is further aimed at the cosmetic, non-therapeutic use of a composition comprising from 0.1 to 49.9% by weight of said extract of shells of cashew nuts according to the invention, relative to the total weight of the composition.

The concentration of the extract in the composition may be selected based on the desired intensity of the exfoliating effect and the intended application format, as follows: for daily care and maintenance exfoliation (leave-on creams or lotions), concentrations of 0.1% to 2% by weight are suitable, as demonstrated in Examples 2.1 (1%) and 2.2 (0.25%); for regular treatment and anti-aging serums (leave-on night serums), concentrations of 2% to 15% are suitable, as demonstrated in Example 2.5 (10%); for intensive exfoliation and spot treatment (rinse-off masks), concentrations of 15% to 30% are suitable, as demonstrated in Examples 2.4 (16%) and 2.3 (30%); and for maximum-intensity peel masks and callus treatments (short-contact rinse-off applications), concentrations of 30% to 49.9% may be used, with the 30% rinse-off peel mask of Example 2.3 providing the baseline for this range. A person of ordinary skill in the art will recognize that the extract concentration can be adjusted within the full claimed range without undue experimentation in light of the teaching provided herein.

According to a first variant, the composition according to the invention is an essentially oily composition.

According to the first variant, said composition comprises from 0.1 to 99.9% by weight of a vegetable oily phase comprising at least one vegetable oil and/or at least one ester of vegetable fatty substance relative to the total weight of the composition.

According to an embodiment, the vegetable oily phase comprises at least one vegetable oil chosen from the group formed by sunflower oil, rapeseed oil, olive oil, camelina oil, peanut oil, coconut oil, grape seed oil, castor oil, argan oil, Djansang oil, desert date oil, hemp oil, Nigella oil, Barbary fig oil, macadamia oil, soybean oil, palm and palm tree oil, Tamanu oil, Kukui nut oil (or candle nut), sesame oil, linseed oil, walnut oil, hazelnut oil, almond oil, baobab oil, passion fruit oil, Brazil nut oil, hibiscus oil, pumpkin seed oil, Luffa oil, Carapa oil, evening primrose oil, borage seed oil, avocado oil, sea buckthorn oil, apricot kernel oil, cherry kernel oil, apple seed oil, pomegranate oil, jojoba oil, rosehip seed oil, plum kernel oil, shea butter, cocoa butter, Kokum butter, mango butter, Moabi butter, Karanja butter, Tucuma butter, Cupuaçu butter, Buriti butter, Murumuru butter, Kombo butter, Kpangnan butter and triglycerides, in particular of caprylic/capric acids.

According to a second particular variant, the composition according to the invention comprises an O/W Pickering emulsion containing an aqueous phase, a vegetable oily phase and an organically modified phyllosilicate.

Pickering emulsions are emulsions stabilized by solid particles. During the preparation of the emulsion, said solid particles are positioned at the interface between the aqueous phase and the oily phase.

Pickering emulsions used according to the present invention are oil-in-water emulsions, i.e. O/W emulsions, stabilized by a particular clay which is a modified natural phyllosilicate.

Beneficially, the aqueous phase of the Pickering emulsion is present in an amount ranging from 43% to 93%, such as 55% to 70% and for example 58% to 65% by weight relative to the total weight of the composition.

According to an embodiment, the modified natural phyllosilicate comprises a phyllosilicate chosen from the group consisting of vermiculites and smectites. For example, the phyllosilicate may be chosen from the group consisting of montmorillonites, bentonites, nonytronites, beidellites, volkonskoites, hectorites, saponites, sauconites, sobockites, stevensites, svinfordites. In an embodiment, the phyllosilicates are sodium, potassium or calcium phyllosilicates, or mixtures thereof. In an embodiment, the phyllosilicate is chosen from the group consisting of hectorite, montmorillonite, bentonite or mixtures thereof.

In an embodiment, the modified natural phyllosilicate comprises an organic compound selected from the group consisting of Xanthan gum, guar gum, Tara gum, locust bean gum, acacia gum, carrageenan, alginate, chitosan, pectin, citric acid, tartaric acid, oxalic acid, succinic acid, malic acid, acetic acid, lactic acid, propionic acid, salicylic acid, glycosaminoglycans. Advantageously, the modified natural phyllosilicate comprises a bentonite modified by Xanthan gum and by citric acid.

According to another embodiment, the modified natural phyllosilicate comprises an organic compound chosen from any organic compound known in the art of Pickering emulsions, such as same disclosed in the French Patent No. FR 2 976 503.

Beneficially, the modified phyllosilicate is present in an amount ranging from 2% to 20%, such as 2.5% to 10% and for example 3.5% to 8% by weight relative to the total weight of the composition.

Beneficially, the oily phase of the Pickering emulsion is present in an amount ranging from 5% to 45%, such as 10% to 35% and for example 15% to 30% by weight relative to the total weight of the composition.

Beneficially, the O/W Pickering emulsion is prepared by a method which comprises at least the following steps in the following order:
preparing the aqueous phase;
adding the organically modified phyllosilicate to the aqueous phase and mixing;
adding the vegetable oily phase to the mixture obtained and obtaining an O/W Pickering emulsion.

Beneficially, the composition according to the present invention comprises at least one cosmetic agent chosen from humectants, thickeners, texturing agents, emulsifiers, dispersing agents, foaming agents, emollients, preservatives, colorants, plant extracts, plant fibers, minerals, pH adjusting agents, active ingredients and perfumes.

Beneficially, the composition according to the invention comprises 0.001 to 20% by weight of at least one cosmetic agent, with respect to the total weight of the composition.

A person skilled in the art will naturally take care to choose such cosmetic agents so as not to alter the properties of the composition according to the invention.

The composition according to the invention may constitute a massage or skin care composition, and in particular a cleanser cream, a protective cream, a treatment cream or a care cream for the face, for the hands, for the feet, for the body, for the scalp or for the nails. More particularly, the composition may be a care cream, a day cream, a night cream, a make-up remover cream, a foundation cream; a fluid foundation, a make-up remover milk, a protective body milk or a care body milk; a lotion, a gel or a foam for the care or hygiene of the skin or of the scalp or of the nails, such as a micellar cleansing lotion.

### Uses

More particularly, an aspect of the present invention is aimed at the cosmetic, non-therapeutic use by, topical route, of said extract or of said composition for its effects on anti-aging, anti-wrinkle and radiance of the complexion and/or anti-spot effects.

More precisely, an aspect of the present invention is aimed at the cosmetic, non-therapeutic use by topical route, of said extract or of said composition for erasing hyperpigmentation spots from the skin and/or improving the radiance of the skin.

More particularly, hyperpigmentation spots on the skin correspond to the accumulation of melanin on the surface of the skin; improving the radiance of the skin corresponds to the elimination of dead skin that diffracts light giving a dull effect to the surface of the skin.

An aspect of the present invention is further aimed at the cosmetic, non-therapeutic use, by topical route of said extract or of said composition for making the skin look smoother, clearer and/or more uniform.

Properties and benefits of the composition according to the present invention include:
removing wrinkles and fine lines by smoothing the skin microrelief,
removing calluses, i.e. protruding calluses, from hands and feet;
removing pigment spots without affecting the hair or degrading the keratin of body hair, of the hair and of the nails.

More particularly, the exfoliating properties of the composition according to the present invention make it possible to remove dead skin around the nails without affecting the nails, to remove dead skin from the skin and from the scalp (dandruff), and in doing so the composition leads to brightening the complexion (removes the dull appearance imparted by dead skin) and/or to make the scalp healthier.

The exfoliating properties of the composition according to an aspect of the present invention also result in lightening the complexion by releasing the accumulation of melanin in the corneocytes, in making the skin healthier by cleansing and removing dead skin (nutrient substrate for microorganisms), in stimulating the cell regeneration of the skin, and in ending with a fresh skin which looks younger.

The composition according to an aspect of the present invention is further used to stimulate the production of collagen and elastin: the invention leads to faster cell renewal and to a stimulation of the basal layer of the epidermis. Such stimulation induces an increased production of collagen, of elastin and of structural proteins of the skin, which gives the skin firmness and elasticity.

An aspect of the present invention is further aimed at a cosmetic, non-therapeutic use, by topical route, of a composition according to the invention as an agent apt to stop superficial bleeding in the event of cuts, in particular following shaving, hair removal or skin scraping.

The following examples are aimed at illustrating the present invention but do not limit the scope of the invention.

### EXAMPLES

### A—Example 1.1—Extract of Shells of Cashew Nuts

### Preparation of the Extract

The shells of cashew nuts have been mechanically cut using a knife mill so as to obtain shell particles of 5 mm or less.

100 g of cut shells of cashew nuts were then fed into a reactor with 300 g of ethanol at a concentration of about 96° v/v (approximately 192 U.S. proof). The term about is used herein to encompasses variations of +/−5%.

Shearing was then maintained under a 4-blade Misceo stirrer for 2 hours at a temperature of about 40° C. The temperature of the mixture should not exceed 45° C., in particular to prevent the conversion of anacardic acid to cardanol.

At the end of the 2 hours, the mixture was filtered through a pleated filter.

243.55 g of filtrate were obtained, i.e. a yield of 60.89%. The extract obtained (filtrate) comprises a dry matter content of 11.17%, i.e. 27.20 g.

The filtrate was then placed in a rotary evaporator at 50° C. where the evaporation of ethanol took place. After the evaporation of ethanol (206.0 g), 4.6 g of concentrated extract of cashew shells was obtained.

### Determination of the Composition of the Extract

By gas chromatography analysis, it was possible to show that the extract obtained comprises 73% of 6-[8(Z)-n-pentadecenyl] salicylic acid, corresponding to formula I with CH₂R equal to C15:1. This 73% figure represents the predominant single component within the anacardic acid fraction. Total anacardic acid content (comprising all four C15 derivatives) of the concentrated extract is consistent with the range of 60% to 95% by weight.

### B—Example 1.2 Histological Study

A study of slides of human skin exposed to an extract of shells of cashew nuts according to the invention, along with a histological analysis after standard H&E staining, was carried out.

Table 1 shows the gel (example 1.2) comprising an O/W Pickering emulsion used in the histological study.

TABLE 1

| Phase | Commercial name | % | INCI |
|---|---|---|---|
| A | Water | Q.S. | Aqua |
| A | Sodium benzoate | 0.40 | Sodium benzoate |
| B | Frametime LTX | 3.5 | Sodium Magnesium Silicate & |
| | marketed by Ephyla | | Xanthan gum & citric acid |
| C | Concentrated | 30.00 | Anacardium Occidentale L |
| | cashew shell | | (Cashew shell) extract |
| | extract obtained | | |
| | in Example 1.1 | | |
| Total | | 100 | |

The emulsion was obtained by solubilizing the preservative “Sodium Benzoate” in water, then mixing under shear at 4500 rpm with a rotor-stator, for 7 minutes, the Frametime LTX so as to obtain a viscous gel. Finally, the extract obtained according to example 1.1 (with oily affinity) was incorporated under shearing at 3500 rpm for 5 minutes so as to form a Pickering emulsion wherein the extract represented the dispersed phase.

The control corresponds to a slide of human skin treated with 500 mg of physiological saline. The other 4 samples, treated and evaluated at different lengths of time, corresponded to a slide of human skin treated with 500 mg of gel (Example 1.2).

Five time points were evaluated: control (untreated), treated sample evaluated at 30 minutes, treated sample evaluated at 60 minutes, treated sample evaluated at 120 minutes and treated sample evaluated at 240 minutes.

All dermal and epidermal structures were evaluated.

Table 2 shows the evolution of observed skin alterations.

TABLE 2

| | Observations on | |
|---|---|---|
| Time | epidermis | Observations on dermis |
| Control | Normal, no special signs | Normal inflammatory cells |
| Sample at | Same as control | No change |
| 30 minutes | | |
| Sample at | Epidermis stays normal | Possible superficial |
| 60 minutes | | elastosis |
| Sample at | Vacuolar altered nuclei | Possible superficial |
| 120 minutes | | elastosis |
| Sample at | Detachment of epidermal | Denser dermis |
| 240 minutes | cells, intact stratum | |
| | corneum | |

The report showed that after 4 hours (240 minutes), the treated sample showed detachment (exfoliation) of epidermal cells, without any exfoliation of dermal cells (absence of necrotic dermal damage).

Said study found proof of a targeted exfoliation, limited to the epidermis.

### B—Example 2—Cosmetic Compositions

### Example 2.1

Table 3 shows a cream according to the invention comprising an O/W Pickering emulsion.

TABLE 3

| Phase | Commercial name | % | INCI |
|---|---|---|---|
| A | Water | Q.S. | Aqua |
| A | Sodium benzoate | 0.40 | Sodium benzoate |
| B | Frametime CXG | 5.0 | Bentonite & Xanthan gum & |
| | marketed by Ephyla | | sodium stearoyl glutamate & |
| B | Amisoft HS-11P | 0.60 | Sodium stearoyl glutamate |
| B | Xanthan gum | 0.10 | Xanthan gum |
| | FF marketed by | | |
| | Jungbunzlauer | | |
| C | Refined sunflower | 25.00 | Helianthus annuus seed oil |
| | oil marketed by | | |
| | CAUVIN | | |
| D | Concentrated | 1.00 | Anacardium Occidentale L |
| | cashew shell | | (Cashew shell) extract |
| | extract obtained | | |
| | in Example 1.1 | | |
| Total | | 100 | |

On the one hand, in order to obtain a homogenized mixture, the constituents of phases A and B were mixed at 20° C. with a mechanical mixer with blades or a rotor stator tool at a speed comprised between 3500 and 4000 rpm (revolutions per minute) for 10 minutes. On the other hand, the constituents of phases C and D were mixed at 20° C. under slow stirring with a single mixing blade. Finally, in order to obtain a homogenized composition, the two mixtures obtained were combined and mixed with a mechanical mixer with blades or a rotor stator tool at a speed of 4000 rpm for 10 minutes.

The pH of the composition obtained was set to 5.

The composition was then used in a “leave on” application.

Said composition was applied in the morning to the body or to the face.

After about three weeks of use, the testers noticed a lighter skin tone.

During the same period, testers also note an erasure of hyperpigmentation spots leading to a lightening of the surface of the skin.

Moreover, the composition was applied to the scalp 3 times a week for 3 weeks, in the evening (the day before shampooing).

From the first week of use the testers noted a disappearance of itching and desquamation (dandruff) of the scalp.

The composition made the scalp healthier and reduced dandruff.

### Example 2.2

Table 4 shows a cream according to the invention.

TABLE 4

| Phase | Commercial name | % | INCI |
|---|---|---|---|
| A | Water | Q.S. | Aqua |
| B | Glycerin | 3.50 | glycerin |
| B | Xanthan gum | 0.10 | Xanthan gum |
| | FF marketed by | | |
| | Jungbunzlauer | | |
| C | Cutina GMS | 4.50 | Glyceryl monostearate |
| C | Cetearyl alcohol | 1.00 | Ceterayl alcohol |
| C | Amisoft HS-11P | 0.30 | Sodium stearoyl glutamate |
| C | Refined sunflower | 25.00 | Helianthus annuus seed oil |
| | oil marketed by | | |
| | CAUVIN | | |
| D | Concentrated | 0.25 | Anacardium Occidentale L |
| | cashew shell | | (Cashew shell) extract |
| | extract obtained | | |
| | in Example 1.1 | | |
| E | Citric acid | 0.40 | Citric acid |
| | monohydrate | | |
| Total | | 100 | |

On the one hand, phase B was mixed with the mixing blade, at room temperature, until the Xanthan gum was dissolved.

On the other hand, phase C was brought to 80° C. in order to be melted.

Phase A was then heated to 45° C., and phase C, constantly maintained at 80° C., was added to phase A.

The mixture of phases (A and C) was then emulsified with phase B using a mixer or a rotor-stator at a speed of 3000 to 4500 rpm until homogenization, for about 7 minutes.

The emulsion was then cooled to 40° C. and phase D was added with slow stirring with a single mixing blade; finally, phase E was added with a rotor-stator at a speed of 3000 to 4500 rpm for 5 minutes.

The pH of the composition obtained is set to 5.

The composition is used in a “leave on” application.

The composition was applied in the morning and in the evening to the body or to the face.

After about three weeks of use, the testers noticed a lighter skin tone.

During the same period, testers also noted an erasure of hyperpigmentation spots leading to a lightening of the surface of the skin.

Moreover, the composition was applied to the scalp 3 times a week for 3 weeks, in the evening (the day before shampooing).

After the period of use, the testers noted the disappearance of itching and desquamation (dandruff) of the scalp.

The composition made the scalp healthier and reduced dandruff.

### Example 2.3

Table 5 shows a body and face peel mask according to the invention comprising an O/W Pickering emulsion.

TABLE 5

| Phase | Commercial name | % | INCI |
|---|---|---|---|
| A | Water | Q.S. | Aqua |
| A | Frametime CX | 4.50 | Bentonite & Xanthan gum & |
| | marketed by Ephyla | | citric acid |
| A | Xanthan gum | 0.10 | Xanthan gum |
| | FF marketed by | | |
| | Jungbunzlauer | | |
| B | Refined sunflower | 25.00 | Helianthus annuus seed oil |
| | oil marketed by | | |
| | CAUVIN | | |
| B | Concentrated | 30.00 | Anacardium Occidentale L |
| | cashew shell | | (Cashew shell) extract |
| | extract obtained | | |
| | in Example 1.1 | | |
| C | Novasolve DMI | 3.00 | Dimethyl isosorbide |
| C | Propanediol | 6.00 | Propanediol |
| C | Sorbitol | 0.80 | Sorbitol |
| Total | | 100 | |

The constituents of phase A were mixed at 20° C. with a mechanical mixer with blades or a rotor stator tool at a speed between 3500 and 4000 rpm until homogenization, for about 7 minutes.

Phase A was then mixed with the constituents of phase B at 20° C. with a mechanical mixer with blades or rotor stator at a speed comprised between 3500 and 4000 rpm until homogenization, for about 5-7 minutes.

Finally, the phase was added at 20° C. with slow stirring with a mechanical mixer with blades or a rotor stator tool at a speed comprised between 2000 and 2500 rpm for 4 to 5 minutes.

The pH of the composition obtained was adjusted to 6.5-7.5.

The mask composition was applied in a thick layer for 10 to 60 minutes and then rinsed with clear water.

On the face, right from the first application, a peeling effect of the corneocytes was observed, i.e. an abrasion of the skin relief and a lighter skin tone.

On the face, the testers noted, right from the first application, an erasure of hyperpigmentation spots leading to a lightening of the surface of the skin.

Around the nails, the product was applied in a thick layer for between 30 and 45 minutes, then rinsed with clear water. The dead skin disappeared when rinsed by lightly rubbing with a wooden spatula; the periphery of the nails looked very clean and neat without any intermediate skin.

On the outer face of the heel of the feet, at the “callus” areas, the product was applied in a thick layer between 30 and 45 minutes, then rinsed with clear water. The “callus” disappeared when rinsed by lightly rubbing with a wooden spatula; the callus gave way to a supple and much more tender skin.

The composition according to the invention provided an anti-aging effect by smoothing, right from the first application.

### Example 2.4

Table 6 shows a body and face peel “leave on” mask according to the invention without aqueous phase. The product is also used as an aftershave in order to stop small bleeding, and to calm razor burn.

TABLE 6

| Phase | Commercial name | % | INCI |
|---|---|---|---|
| A | OLO3D marketed | 14.00 | Stearalkonium Bentonite |
| | by Ephyla | | |
| B | Refined sunflower | 57.00 | Helianthus annuus seed oil |
| | oil marketed by | | |
| | CAUVIN | | |
| B | Moringa oil | 5.00 | Moringa oleifera seed oil |
| | marketed by Ephyla | | |
| B | Organic desert | 5.00 | Balanites roxburghii seed oil |
| | date oil marketed | | |
| | by Ephyla | | |
| B | HTRE marketed | 3.00 | Non-volatile Helianthus annuus |
| | by Ephyla | | seed oil & Canarium luzonicum |
| | | | gum |
| C | Concentrated | 16.00 | Anacardium Occidentale L |
| | cashew shell | | (Cashew shell) extract |
| | extract obtained | | |
| | in Example 1.1 | | |
| Total | | 100 | |

In order to obtain a homogenized mixture, the constituents of phases A and B were mixed at 20° C. with a mechanical mixer with blades or a rotor stator tool at a speed comprised between 3500 and 4000 rpm, for 7 minutes. In order to obtain a homogenized composition, phase C was then added at 20° C. and mixed with a mechanical mixer with blades or a rotor stator tool at a speed comprised between 3500 and 4000 rpm (for about 5-7 minutes).

The composition was used as a “leave on” application directly on the spots in order to target the spots or as an aftershave to stop minor superficial bleeding and or to soothe razor burn.

A plumping effect was observed, which smoothens the surface of the skin and brings a beautiful radiance to the complexion. The composition according to the invention provides an anti-aging effect by smoothing.

As an aftershave, the composition very quickly stops small bleeding and relieves razor burn.

### Example 2.5

Table 7 shows a night serum according to the invention comprising an O/W Pickering emulsion.

TABLE 7

| Phase | Commercial name | % | INCI |
|---|---|---|---|
| A | Water | Q.S. | Aqua |
| A | Glycerin | 3.00 | glycerin |
| A | Sodium benzoate | 0.40 | Sodium benzoate |
| B | Frametime CXG | 5.0 | Bentonite & Xanthan gum & |
| | marketed by | | sodium stearoyl glutamate & |
| | Ephyla | | citric acid |
| B | Amisoft HS-11P | 0.60 | Sodium stearoyl glutamate |
| B | Xanthan Gum | 0.15 | Xanthan gum |
| | FF marketed by | | |
| | Jungbunzlauer | | |
| C | HTRE marketed | 2.50 | Non-volatile Helianthus annuus |
| | by Ephyla | | seed oil & Canarium luzonicum |
| | | | gum |
| C | Refined sunflower | 12.50 | Helianthus annuus seed oil |
| | oil marketed by | | |
| | CAUVIN | | |
| D | Concentrated | 10.00 | Anacardium Occidentale L |
| | cashew shell | | (Cashew shell) extract |
| | extract obtained | | |
| | in Example 1.1 | | |
| E | Citric acid | 0.40 | Citric acid |
| | monohydrate | | |
| Total | | 100 | |

On the one hand, in order to obtain a homogenized mixture, the constituents of phases A and B were mixed at 20° C. with a mechanical mixer with blades or a rotor stator tool at a speed comprised between 3500 and 4000 rpm, for 7 minutes. On the other hand, the constituents of phases C and D were mixed at 20° C. under slow stirring with a single mixing blade. Finally, the two mixtures obtained were combined and mixed with a mechanical mixer with blades or a rotor stator tool at a speed comprised between 3000 and 4500 until homogenization, for 5-7 minutes.

The pH of the composition obtained was set to 5 by means of phase E.

The composition was applied in a thin layer after cleansing the skin with clear water, then left overnight until morning cleansing also with clear water.

A peeling effect of corneocytes was observed, that is an abrasion of the skin relief and a lighter skin tone over a week of application in the evening.

The testers noted an erasure of hyperpigmentation spots leading to a lightening of the surface of the skin over a week of application in the evening.

The composition according to the invention provided an anti-aging effect by smoothing.

Users have noted an exfoliating effect on the surface of the skin (epidermis) noticeable right from the first applications and, in particular, anti-pigmentary spot properties.

The extract of shells of cashew nuts (Anacardium occidentale L.) used in accordance with one or more aspects of the present invention is derived from a natural, plant-based source. Unlike conventional chemical peel agents such as alpha-hydroxy acids (AHAs), which require formulation in an acidic medium (low pH) to exert their keratolytic activity, the extract of the present invention is active as an exfoliating agent without the need for an acidic vehicle. Although the extract remains effective at acidic pH, its activity is not pH-dependent in the same manner as AHAs. As a result, the extract and compositions of the present invention can be formulated at a broader range of pH values, including mildly acidic and near-neutral pH, thereby reducing or eliminating the tingling, irritation, and discomfort that are commonly associated with acid-based peel formulations. This makes the extract and compositions of the present invention particularly well-suited for use by individuals with sensitive or reactive skin, who may not tolerate conventional AHA-based exfoliating treatments.

The exfoliating activity of the extract and compositions of the present invention has been confirmed by histological analysis of human skin specimens. Upon application of a composition comprising the extract to human skin ex vivo, progressive epidermal changes were observed over a period of up to 240 minutes, culminating in the detachment of epidermal cells with the stratum corneum remaining intact. Critically, no necrotic damage to dermal structures was observed at any time point evaluated. This histological evidence demonstrates that the exfoliating action of the extract is targeted and confined to the epidermis, and does not extend to the dermis. This selective, superficial mode of action distinguishes the present invention from more aggressive resurfacing treatments and supports its use as a safe, cosmetic, non-therapeutic exfoliating agent.

The methods and compositions of the present invention provide measurable improvements in complexion uniformity and skin radiance. Dead skin cells present on the surface of the epidermis diffract light, imparting a dull appearance to the skin. By removing these dead cells through the exfoliating action of the extract, the compositions of the present invention restore a brighter, more radiant skin surface. These benefits were observed in human use testing of compositions according to the invention at extract concentrations ranging from 0.25% to 30% by weight, with effects observed as early as the first application at higher concentrations and after approximately three weeks of daily use at lower maintenance concentrations.

By stimulating the natural cell renewal process of the epidermis, the extract and compositions of the present invention provide anti-aging benefits. With aging, the rate of natural epidermal cell renewal slows, leading to an accumulation of dead skin cells that contributes to a rougher skin texture and the more pronounced appearance of fine lines and wrinkles. By accelerating the removal of these dead cells and stimulating the basal layer of the epidermis, the extract promotes faster cell turnover, which in turn stimulates the production of collagen, elastin, and other structural proteins of the skin. The resulting increase in collagen and elastin content imparts greater firmness and elasticity to the skin. The smoothing of the skin microrelief through exfoliation further reduces the visible depth of fine lines and wrinkles, providing a rejuvenated appearance. These anti-aging effects were observed in human use testing of compositions according to the invention and were noted from the first application in compositions comprising higher concentrations of the extract.

The cosmetic compositions according to the present invention have been found to be stable, non-irritating, non-toxic, and non-allergenic when applied to the skin. The histological study described herein confirmed that the exfoliating action of the compositions is confined to the epidermis and does not produce necrotic damage to the underlying dermis. Because the extract does not require an acidic formulation vehicle to be active, the compositions of the present invention avoid the pH-associated irritation and discomfort commonly encountered with AHA-based chemical peels, making them suitable for use on sensitive and reactive skin. The compositions are effective in both leave-on and rinse-off application formats, across a wide range of extract concentrations (0.25% to 30% by weight), demonstrating formulation flexibility without compromising the safety profile of the product.

The articles “a” and “an” may be employed in connection with various elements and components of compositions, processes described herein. This is merely for convenience and to give a general sense of the compositions and processes. Such a description includes “one or at least one” of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description is by way of example only.

1. A cosmetic, non-therapeutic method of exfoliating the epidermis of a subject, comprising topically applying to the skin of the subject a cosmetically effective amount of an extract of shells of cashew nuts or “Anacardium Occidentale L.”, wherein said extract of shells of cashew nuts is obtained by a method of preparation comprising at least the following steps, in the following order:
providing shells of cashew nuts,
grinding the shells of cashew nuts to an average particle size of 5 mm or less,
extracting from the ground shells of cashew nuts using ethanol at a concentration of about 96° v/v at a temperature between 20° C. and 45° C. to obtain a resulting mixture,
filtering the resulting mixture,
recovering the filtrate,
evaporating the ethanol from the filtrate, and
recovering the extract obtained.

2. The method according to claim 1, wherein the extract of shells of cashew nuts comprises from 60 to 95% by weight of anacardic acid relative to a total weight of the extract.

3. The method according to claim 1, wherein the extract of shells of cashew nuts comprises from 50 to 90% by weight of 6-[8(Z)-n-pentadecenyl] salicylic acid relative the total weight of the extract.

4. A cosmetic, non-therapeutic method of exfoliating the epidermis of a subject, comprising topically applying to the skin of the subject a cosmetic composition comprising from 0.1 to 49.9% by weight of extract of shells of cashew nuts or “Anacardium Occidentale L.” with respect to a total mass of the composition, wherein said extract is prepared by the method of preparation as defined in claim 1.

5. The method according to claim 4, wherein the composition comprises from 0.1 to 99.9% by weight of a vegetable oily phase comprising at least one vegetable oil and/or at least one vegetable fatty ester relative to the total weight of the composition.

6. The method according to claim 5, wherein the vegetable oily phase comprises at least one vegetable oil chosen from the group formed by sunflower oil, rapeseed oil, olive oil, camelina oil, peanut oil, coconut oil, grape seed oil, castor oil, argan oil, Djansang oil, desert date oil, hemp oil, Nigella oil, Barbary fig oil, macadamia oil, soybean oil, palm and palm tree oil, Tamanu oil, Kukui nut oil (or candle nut), sesame oil, linseed oil, walnut oil, hazelnut oil, almond oil, baobab oil, passion fruit oil, Brazil nut oil, hibiscus oil, pumpkin seed oil, Luffa oil, Carapa oil, evening primrose oil, borage seed oil, avocado oil, sea buckthorn oil, apricot kernel oil, cherry kernel oil, apple seed oil, pomegranate oil, jojoba oil, rosehip seed oil, plum kernel oil, shea butter, cocoa butter, Kokum butter, mango butter, Moabi butter, Karanja butter, Tucuma butter, Cupuaçu butter, Buriti butter, Murumuru butter, Kombo butter, Kpangnan butter and triglycerides.

7. The method according to claim 6, wherein the at least vegetable oil is formed by caprylic/capric acids.

8. The method according to claim 4, wherein said composition comprises an O/W Pickering emulsion containing an aqueous phase, a vegetable oil phase and an organically modified phyllosilicate.

9. The method according to claim 8, wherein the organically modified phyllosilicate is present in an amount ranging from 2% to 20% by weight relative to the total weight of the composition.

10. The method according to claim 9, wherein the organically modified phyllosilicate is present in an amount ranging from 2.5% to 10% by weight relative to the total weight of the composition.

11. The method according to claim 10, wherein the organically modified phyllosilicate is present in an amount ranging from 2.5% to 8% by weight relative to the total weight of the composition.

12. The method according to claim 4, wherein the composition comprises at least one cosmetic agent chosen from humectants, thickeners, texturing agents, emulsifiers, dispersants, foaming agents, emollients, preservatives, colorants, plant extracts, plant fibers, minerals, pH adjusting agents, active ingredients and perfumes.

13. The method according to claim 4, wherein the applying removes hyperpigmentation spots from the skin and/or improves the radiance of the skin.

14. The method according to claim 13, wherein the applying makes the skin look smoother, clearer and/or more uniform.

15. A cosmetic, non-therapeutic method of treatment for exfoliating the epidermis comprising topically applying to the skin of a subject a cosmetically effective amount of extract as defined in claim 1.

16. A cosmetic composition for exfoliating the epidermis, comprising:
(a) from 0.1% to 49.9% by weight of an extract of shells of cashew nuts (“Anacardium Occidentale L.”), relative to the total weight of the composition, wherein said extract is prepared by a method comprising: grinding shells of cashew nuts to an average particle size of 5 mm or less; extracting from the ground shells using ethanol at a concentration of about 96% v/v at a temperature between 20° C. and 45° C.; filtering; recovering the filtrate; evaporating the ethanol; and recovering the extract; and
(b) at least one cosmetically acceptable vehicle.

17. The cosmetic composition according to claim 16, comprising an O/W Pickering emulsion comprising an aqueous phase, a vegetable oil phase, and an organically modified phyllosilicate.
